(19) United States
(12) Patent Application Publication
(10) Pub. No.: US 2020/0215022 A1
(43) Pub. Date: Jul. 9, 2020

(54) NOVEL CANNABINOID COMPOSITIONS AND METHODS OF TREATING PEDIATRIC EPILEPSY

(71) Applicant: TILRAY, INC., Seattle, WA (US)

(72) Inventors: Catherine JACOBSON, Seattle, WA (US); Caleb Joshua EADES, Nanaimo (CA)

(21) Appl. No.: 16/471,407

(22) PCT Filed: Dec. 20, 2017
(86) PCT No.: PCT/IB2017/001683

§ 371 Date: Jun. 19, 2019

Related U.S. Application Data
(60) Provisional application No. 62/436,861, filed on Dec. 20, 2016.

(51) Int. Cl. A61K 31/352; A61K 36/185; A61K 31/05; A61K 47/44; A61P 25/08

(52) U.S. Cl. CPC A61K 31/352; A61K 36/185; A61K 9/0053; A61K 47/44; A61P 25/08; A61K 31/05

(57) ABSTRACT

The present disclosure describes various novel compositions and methods, wherein said compositions and methods comprise CBD and THC in certain relative ratios.

### CROSS-REFERENCE TO RELATED APPLICATIONS

This application claims benefit under 35 U.S.C. § 119(e) of U.S. Provisional Application No. 62/436,861 filed Dec. 20, 2016, the contents of which are incorporated herein by reference in their entirety.

### FIELD

The present disclosure relates to novel compositions of matter, including oral solutions, comprising cannabinoids THC and CBD. The present disclosure further relates to novel methods of use of compositions comprising THC and CBD.

### BACKGROUND

The primary active ingredients in cannabis, THC and CBD, are 21-carbon terpenophenolic compounds. These two compounds were isolated from the Cannabis plant approximately 60 years ago, and their structures and chemical properties have been well characterized (Gaoni and Mechoulam, 1964). There have been extensive reviews on the pharmacology and potential therapeutic potential of THC and CBD (Kreitzer and Stella, 2009; Pertwee et al., 2010).

THC and CBD are generally thought to exert their actions via the endocannabinoid system, although CBD's mechanism of action may include receptors and pathways outside this system (extensively reviewed by Pertwee et al., 2010). The primary cannabinoid receptors include CB1, with a neuromodulatory role and CB2, with an immunomodulatory role (GW Pharma, 2015). Within the brain, the distribution of CB1 receptors is heterogeneous, accounting for several well-documented pharmacological properties of CB1 receptor agonists, such as phytocannabinoids. CBD, while considered part of the cannabinoid family due to its chemical structure, does not appear to have a great affinity for either of the cannabinoid receptors (Pacher et al., 2006). The lack of interaction with the CB1 receptor is thought to explain the well-established safety profile of CBD and lack of psychotropic effect relative to THC.

### SUMMARY

The present disclosure is based, in part, on the surprising discovery that certain compositions of matter comprising the cannabinoids CBD and THC in certain relative ratios may have particular utility, for instance in the treatment or prevention of certain diseases, conditions, or symptoms.

In one embodiment of the disclosure, there is provided a novel composition of matter wherein the composition comprises the cannabinoids CBD and THC. In certain embodiments of the disclosure, the cannabinoids CBD and THC are present in specific relative ratios. In certain embodiments of the disclosure, the relative ratio of CBD to THC in the novel composition is at least 20:1 CBD:THC, preferably 25:1 CBD:THC, more preferably 30:1 CBD:THC, even more preferably 40:1 CBD:THC, or most preferably 50:1 CBD:THC.

It is a feature of the disclosure that the cannabinoids CBD and THC may be present in a medicament or pharmaceutical composition. In one embodiment of the disclosure, there is provided a novel medicament or pharmaceutical composition, wherein the medicament or pharmaceutical preparation comprises the cannabinoids CBD and THC. In certain embodiments of the disclosure, the cannabinoids CBD and THC are present in certain relative ratios. In certain embodiments of the disclosure, the relative ratio of CBD to THC in the novel composition is at least 20:1 CBD:THC, preferably 25:1 CBD:THC, more preferably 30:1 CBD:THC, even more preferably 40:1 CBD:THC, or most preferably 50:1 CBD:THC. The medicament or pharmaceutical composition may further comprise other excipients, carriers, stabilizers, and the like. In certain embodiments of the disclosure, the medicament or pharmaceutical composition further comprises one or more excipients. In certain embodiments, the one or more excipients comprises a carrier substance. The carrier substance may be an oil or lipid based substance. The carrier may be grapeseed oil, coconut oil, medium chain triglycerides (MCT), sesame oil, or similar substance. In certain embodiments, the cannabinoids CBD and THC are formulated in carrier to specific concentrations, and with certain relative ratios. In certain embodiments, the cannabinoids are formulated to a concentration of 100 mg/mL of CBD and 2 mg/mL THC in carrier substance. In certain embodiments of the disclosure, the carrier is grapeseed oil.

In a certain aspect of the disclosure, there is provided a novel method of treatment for a variety of diseases, conditions, or symptoms, wherein the method comprises administering a therapeutically effective amount of a composition, medicament, or pharmaceutical preparation of the present disclosure, as described herein. The disease, condition, or symptom may include, but is not limited to: seizure disorders such as epilepsy, treatment-resistant epilepsy, Dravet Syndrome, Lennox-Gastaut Syndrome; spasticity disorders such as multiple sclerosis; neurodegenerative disorders such as Parkinson's Disease, Alzheimer's Disease, Huntington's Disease or amyloid lateral sclerosis (ALS); proliferative diseases such as cancer; dermatological conditions such as psoriasis; mental health conditions such as post-traumatic stress disorder (PTSD), insomnia, anxiety, depression, or schizophrenia; respiratory diseases such as chronic obstructive pulmonary disorder (COPD).

It should be appreciated that all combinations of the foregoing concepts and additional concepts discussed in greater detail below (provided such concepts are not mutually inconsistent) are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

In one aspect of any of the embodiments, described herein is a pharmaceutical composition comprising cannabidiol (CBD) and delta-9-tetrahydrocannabinol (THC) at a ratio of from about 40:1 to about 60:1. In some embodiments of any of the aspects, the CBD:THC ratio is from about 45:1 to about 55:1. In some embodiments of any of the aspects, the CBD:THC ratio is about 50:1. In some embodiments of any of the aspects, the CBD:THC ratio is 50:1.

In one aspect of any of the embodiments, described herein is a pharmaceutical composition comprising cannabidiol (CBD) and delta-9-tetrahydrocannabinol (THC) at a ratio of from about 40:1 to about 60:1 for use in the treatment of pediatric epilepsy. In some embodiments of any of the aspects, the CBD:THC ratio is from about 45:1 to about 55:1. In some embodiments of any of the aspects, the CBD:THC ratio is about 50:1. In some embodiments of any of the aspects, the CBD:THC ratio is 50:1.

In some embodiments of any of the aspects, the CBD and THC are formulated in grape seed oil. In some embodiments of any of the aspects, the CBD and THC are obtained from Cannabis sativa L.

In some embodiments of any of the aspects, the CBD and THC are provided at concentrations of about 100 mg/mL and 2 mg/mL respectively. In some embodiments of any of the aspects, the composition further comprises one or more pharmaceutically acceptable carriers or excipients.

In one aspect of any of the embodiments, described herein is a method of treating pediatric epilepsy in a subject in need thereof, the method comprising administering a composition described herein to the subject. In some embodiments of any of the aspects, the composition is administered twice daily. In some embodiments of any of the aspects, the composition is administered orally. In some embodiments of any of the aspects, the composition is administered by inhalation.

In some embodiments of any of the aspects, the subject is administered the composition of any of claims 1-8 at a dosage of from about 1 mg/kg/day of CBD to about 18 mg/kg/day of CBD. In some embodiments of any of the aspects, the subject is administered the composition of any of claims 1-8 at a dosage of from about 5 mg/kg/day of CBD to about 18 mg/kg/day of CBD. In some embodiments of any of the aspects, the subject is administered the composition of any of claims 1-8 at a dosage of from about 7 mg/kg/day of CBD to about 16 mg/kg/day of CBD. In some embodiments of any of the aspects, the subject is administered the composition of any of claims 1-8 at a dosage of from 2 mg/kg/day of CBD to 16 mg/kg/day of CBD. In some embodiments of any of the aspects, the subject is administered the composition of any of claims 1-8 at an increasing dosage of the composition, wherein the increase is about 2 mg/kg/day of CBD every 7 days. In some embodiments of any of the aspects, the maximal dose is from about 13 mg/kg/day of CBD to about 14.5 mg/kg/day of CBD. In some embodiments of any of the aspects, the maximal dose is about 16 mg/kg/day of CBD.

In some embodiments of any of the aspects, the subject is administered at least one further concomitant antiepileptic drug (AED). In some embodiments of any of the aspects, the treatment reduces the frequency or severity of seizures.

### DETAILED DESCRIPTION

Following below are more detailed descriptions of various concepts related to, and embodiments of, novel cannabinoid compositions and methods of use. It should be appreciated that various concepts introduced above and discussed in greater detail below may be implemented in any of numerous ways, as the disclosed concepts are not limited to any particular manner of implementation. Examples of specific implementations and applications are provided for illustrative purposes and not by way of limitation.

Any terms not directly defined herein shall be understood to have the meanings commonly associated with them as understood within the art of the disclosure. As employed throughout the specification, the following terms, unless otherwise indicated, shall be understood to have the following meanings.

The term ‘cannabis’ means a genus of flowering plants that includes three putative species, Cannabis sativa, Cannabis indica, and Cannabis ruderalis. The term cannabis may also refer to plant material derived or extracted from the cannabis plant, for instance the leaves, stem, seeds, flowering bodies, or other portions of the plant.

The term ‘cannabinoid’ or ‘cannabinoids’ means a class of chemical compounds which include the phytocannabinoids (oxygen-containing C21 aromatic hydrocarbon compounds found in the cannabis plant), and chemical compounds which mimic the actions of phytocannabinoids or have a similar structure (e.g. endocannabinoids, found in the nervous and immune systems of animals and that activate cannabinoid receptors). Phytocannabinoids are known to occur in significant quantities in the cannabis plant, and may include, but are not limited to tetrahydrocannabinol (THC), cannabidiol (CBD), cannabinol (CBN), and cannabigerol (CBG).

The term ‘THC’ means tetrahydrocannabinol and may include different isoforms and variants, such as delta-9-Tetrahydrocannabinol (Δ9-THC) and delta-8-tetrahydrocannabinol (Δ8-THC).

The term ‘CBD’ means cannabidiol, a cannabinoid often found in cannabis, and having a CAS registry number 13956-29-1. Cannabidiol is known to have many beneficial medicinal qualities, as described elsewhere in this application. In some embodiments, the cannabinoid has the structure of Formula I.

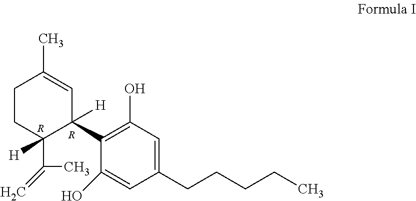

The term ‘therapeutically effective amount’ means a dosage of sufficient quantity to exert a therapeutic effect, to alleviate a symptom, to prevent the onset or progression of a disease, or to cause effective treatment of a disease. A therapeutically effective amount may be determined, for instance, by a dose escalation study, or dose titration.

The term ‘medicament’ means a pharmaceutical composition comprising active and inactive ingredients in sufficient quantities to exert a medically beneficial effect. A medicament may comprise a therapeutically effective amount of active ingredients. A medicament may further comprise additional excipients, additives, stabilizers, carriers, or other compounds to improve the formulation, stability, bioavailability, pharmacokinetics, pharmacodynamics, or other properties of the medicament. The medicament of the present disclosure comprises therapeutically effective amounts of the cannabinoids THC and CBD in certain relative ratios. In certain embodiments, CBD is the predominant cannabinoid, with relatively lower amounts of THC. In certain embodiments, the CBD to THC ratio is greater than 20 to 1. In other embodiments, the CBD to THC ratio is greater than 25 to 1. In other embodiments, the CBD to THC ratio is greater than 30 to 1. In other embodiments, the CBD to THC ratio is greater than 40 to 1. In other embodiments, the CBD to THC ratio is greater than 50 to 1. In certain embodiments, the medicament of the present disclosure comprises a nonpolar carrier oil such as grapeseed oil, coconut oil, medium chain triglycerides (MCT), sesame oil, or another carrier oil. In certain embodiments of the disclosure, the carrier oil is grapeseed oil. In certain embodiments of the disclosure, the medicament of the present disclosure comprises CBD in a concentration of 100 mg/mL and THC in a concentration of 2 mg/mL in grapeseed oil.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In one aspect of any of the embodiments, described herein is a composition comprising cannabidiol (CBD) and delta-9-tetrahydrocannabinol (THC) at a ratio of from about 40:1 to about 60:1. The ratio of CBD:THC can be, e.g., from about 40:1 to about 60:1, from about 45:1 to about 55:1, from about 48:1 to about 52:1, from 40:1 to 60:1, from 45:1 to 55:1, from 48:1 to 52:1, about 50:1, or 50:1. In some embodiments of any of the aspects, the compositions described herein can be pharmaceutical compositions comprising CBD and THC in the specified ratios and further comprising one or more pharmaceutically acceptable carriers or excipients. Compositions comprising the ratios of CBD and THC described herein provide the surprising effect of reducing seizure frequency and/or severity in pediatric epileptic patients with a low incidence of side effects, e.g., increases in blood pressure, dizziness, irritability, ataxia, blurred vision, diplopia vision, rashes, motor incoordination/falls, gastrointestinal (anorexia, loss of appetite, nausea, vomiting, and weight loss), aggression, hostility, irritability, anger, and homicidal ideation/threats, which are observed with prior art formulations. The compositions described herein comprise CBD and THC, which can be obtained from cannabis, e.g., Cannabis sativa L. The CBD and THC can be isolated and/or extracted from cannabis by methods known in the art and formulated by any method known in the art. In some embodiments of any of the aspects, the CBD and THC are formulated in oil, e.g., a plant/vegetable oil, grape seed oil.

In some embodiments of any of the aspects, the compositions described herein can be plant extracts, e.g., whole plant extracts. In some embodiments of any of the aspects, the compositions described herein can be purified compositions.

In some embodiments of any of the aspects, the composition described herein comprises CBD and THC at concentrations of from about 80 mg/mL to about 120 mg/mL and from about 1 mg/mL to about 4 mg/mL, respectively; from about 90 mg/mL to about 110 mg/mL and from about 1.5 mg/mL to about 3 mg/mL, respectively; from about 96 mg/mL to about 104 mg/mL and from about 1.75 mg/mL to about 2.5 mg/mL, respectively; from 80 mg/mL to 120 mg/mL and from 1 mg/mL to 4 mg/mL, respectively; from 90 mg/mL to 110 mg/mL and from 1.5 mg/mL to 3 mg/mL, respectively; from 96 mg/mL to 104 mg/mL and from 1.75 mg/mL to 2.5 mg/mL, respectively; about 100 mg/mL and about 2 mg/mL respectively; or 100 mg/mL and 2 mg/mL respectively.

It is understood by one of skill in the art that active pharmaceutical ingredients may be found in concentrations that vary from the labelled assay specification. The active ingredients in the medicaments of the present disclosure may thus be expected to vary between at least 80% to 120% of the specified concentration. In further embodiments, the active ingredients in the medicaments of the present disclosure may be expected to vary between at least 90% to 110% of the specified concentration.

In one aspect of any of the embodiments, described herein is a method of treating pediatric epilepsy in a subject in need thereof, the method comprising administering a composition as described herein to the subject, e.g., a composition comprising cannabidiol (CBD) and delta-9-tetrahydrocannabinol (THC) at a ratio of from about 40:1 to about 60:1. In some embodiments of any of the aspects, the methods described herein reduce the frequency and/or severity of seizures. In some embodiments of any of the aspects, the methods described herein comprise administering an amount and/or number of doses of the described compositions which are effective to reduce the frequency and/or severity of seizures with a low incidence of side effects.

In some embodiments, the methods described herein relate to treating a subject having or diagnosed as having epilepsy (e.g., pediatric epilepsy) with a composition described herein. Subjects having pediatric epilepsy can be identified by a physician using current methods of diagnosing pediatric epilepsy. Symptoms and/or complications of pediatric epilepsy which characterize these conditions and aid in diagnosis are well known in the art and include but are not limited to, staring, seizures, tremors, stiffening of the body, loss of consciousness, breathing problems, lack of response to noise, apparent confusion, extreme sleepiness and irritability upon waking, head nodding, vomiting, changes in vision in speech, and periods of rapid blinking. Tests that may aid in a diagnosis of, e.g. epilepsy include, but are not limited to, blood tests, electroencephalogram (EEG), CT, MRI, or PET of the brain, and DNA testing for genetic causes. A family history of epilepsy, or exposure to risk factors for pediatric epilepsy (e.g. head injury, brain tumor, trauma, stroke, or certain metabolic problems) can also aid in determining if a subject is likely to have epilepsy or in making a diagnosis of epilepsy.

In some embodiments of any of the aspects described herein, the subject is a pediatric subject. A “subject in need” of treatment for a particular condition can be a subject having that condition, diagnosed as having that condition, or at risk of developing that condition.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

In some embodiments of any of the aspects, the compositions described herein can be administered orally, e.g., as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

In some embodiments of any of the aspects, the compositions described herein can be administered by inhalation, e.g., as a vapor or aerosol formulation or by nebulization. For use as aerosols, a composition described herein can be provided in solution or suspension may be packaged in a pressurized aerosol container together with suitable propellants, for example, hydrocarbon propellants like propane, butane, or isobutane with conventional adjuvants. A composition described herein can also be administered in a non-pressurized form such as in a nebulizer or atomizer. In some embodiments, a composition can also be administered directly to the airways in the form of a dry powder, e.g., by use with an inhaler. Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to twice daily depending on a number of clinical factors, such as the subject's sensitivity to the therapy. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. A composition described herein can be administered over a period of time, such as over a 5 minute, 10 minute, 15 minute, 20 minute, or 25 minute period.

Examples of dosing and/or treatment schedules can include weekly, every other day, daily, twice daily, thrice daily, or more frequent administration. In some embodiments of any of the aspects, the compositions described herein are administered daily. In some embodiments of any of the aspects, the compositions described herein are administered twice daily.

In some embodiments of any of the aspects, the daily dosage of the compositions described herein is from about 1 mg/kg/day of CBD to about 18 mg/kg/day of CBD. In some embodiments of any of the aspects, the daily dosage of the compositions described herein is from 1 mg/kg/day of CBD to 18 mg/kg/day of CBD. In some embodiments of any of the aspects, the daily dosage of the compositions described herein is from about 5 mg/kg/day of CBD to about 18 mg/kg/day of CBD. In some embodiments of any of the aspects, the daily dosage of the compositions described herein is from 5 mg/kg/day of CBD to 18 mg/kg/day of CBD. In some embodiments of any of the aspects, the daily dosage of the compositions described herein is from about 7 mg/kg/day of CBD to about 18 mg/kg/day of CBD. In some embodiments of any of the aspects, the daily dosage of the compositions described herein is from 7 mg/kg/day of CBD to 18 mg/kg/day of CBD.

In some embodiments of any of the aspects, the daily dosage of the compositions described herein is from about 2 mg/kg/day of CBD to about 16 mg/kg/day of CBD. In some embodiments of any of the aspects, the daily dosage of the compositions described herein is from 2 mg/kg/day of CBD to 16 mg/kg/day of CBD.

In some embodiments of any of the aspects, the dosage of the composition administered to the subject increases over time, e.g., from a dose of about 1 mg/kg/day of CBD to about 3 mg/kg/day of CBD to a maximal dose. In some embodiments of any of the aspects, the dosage of the composition administered to the subject increases over time, e.g., from a dose of 1 mg/kg/day of CBD to 3 mg/kg/day of CBD to a maximal dose. In some embodiments of any of the aspects, the dosage is increased at a rate of about 2 mg/kg/day of CBD every 7 days. In some embodiments of any of the aspects, the dosage is increased at a rate of 2 mg/kg/day of CBD every 7 days.

In some embodiments of any of the aspects, the maximal dosage of the compositions described herein is from about 11 mg/kg/day of CBD to about 18 mg/kg/day of CBD. In some embodiments of any of the aspects, the maximal dosage of the compositions described herein is from 11 mg/kg/day of CBD to 18 mg/kg/day of CBD. In some embodiments of any of the aspects, the daily dosage of the compositions described herein is from about 13 mg/kg/day of CBD to about 14.5 mg/kg/day of CBD. In some embodiments of any of the aspects, the daily dosage of the compositions described herein is from 13 mg/kg/day of CBD to 14.5 mg/kg/day of CBD. In some embodiments of any of the aspects, the maximal dosage of the compositions described herein is about 16 mg/kg/day of CBD. In some embodiments of any of the aspects, the maximal dosage of the compositions described herein is 16 mg/kg/day of CBD.

The dosage ranges for the administration of the compositions described herein, according to the methods described herein depend upon, for example, the form of the composition, its potency, and the extent to which symptoms, markers, or indicators of a condition described herein are desired to be reduced, for example the percentage reduction desired for seizure frequency or severity. The dosage should not be so large as to cause adverse side effects, as described elsewhere herein. Generally, the dosage will vary with the age, condition, and sex of the patient and can be determined by one of skill in the art. The dosage can also be adjusted by the individual physician in the event of any complication.

The efficacy of a composition described in, e.g. the treatment of a condition described herein, or to induce a response as described herein (e.g. reduction in seizures) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. seizure frequency or the markers described in the Examples herein. Efficacy can also be measured by a failure of an individual to worsen as assessed by hospitalization, or need for medical interventions (i.e., progression of the disease is halted). Methods of measuring these indicators are known to those of skill in the art and/or are described herein.

Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease.

In some embodiments of any of the aspects, the subject can be receiving and/or administered additional therapies and/or therapeutic agents. In some embodiments of any of the aspects, the subject is administered at least one further antiepileptic drug (AED), e.g., concurrently, concomitantly, previously, or subsequently. Antiepipleptic drugs or anticonvulsants, are known in the art and can include, without limitation, Acetazolamide, Carbamazepine, Clobazam, Clonazepam, Eslicarbazepine acetate, Ethosuximide, Gabapentin, Lacosamide, Lamotrigine, Levetiracetam, Nitrazepam, Oxcarbazepine, Perampanel, Piracetam, Phenobarbital, Phenytoin, Pregabalin, Primidone, Rufinamide, Sodium valproate, Stiripentol, Tiagabine, Topiramate, Vigabatrin, and Zonisamide.

### EXAMPLE EMBODIMENT

The inventors herein describe novel compositions of matter comprising cannabinoids CBD and THC in certain relative ratios. In certain embodiments of the disclosure, there are provided novel compositions of matter comprising CBD and THC in certain relative ratios. In one embodiment, the CBD to THC ratio is at least 20 to 1. In one embodiment, the CBD to THC ratio is at least 25:1. In one embodiment, the CBD to THC ratio is at least 30 to 1. In one embodiment, the CBD to THC ratio is at least 40 to 1. In one embodiment, the CBD to THC ratio is at least 50 to 1.

The present disclosure provides novel medicaments, or pharmaceutical compositions that are beneficial for the treatment or prevention of a disease or condition, or for the alleviation of symptoms of a disease or condition. In certain embodiments, the medicament comprises THC and CBD in certain relative ratios. In one embodiment, the CBD to THC ratio is at least 20 to 1. In one embodiment, the CBD to THC ratio is at least 25 to 1. In one embodiment, the CBD to THC ratio is at least 30 to 1. In one embodiment, the CBD to THC ratio is at least 40 to 1. In one embodiment, the CBD to THC ratio is at least 50 to 1.

The present disclosure also provides novel methods of treatment using the disclosed novel compositions of the present disclosure. In one embodiment, there is provided a method of treatment or prevention of disease, wherein the method comprises administering a therapeutically effective amount of a medicament comprising CBD and THC in certain relative ratios. In one embodiment, the CBD to THC ratio is at least 20 to 1. In one embodiment, the CBD to THC ratio is at least 25:1. In one embodiment, the CBD to THC ratio is at least 30 to 1. In one embodiment, the CBD to THC ratio is at least 40 to 1. In one embodiment, the CBD to THC ratio is at least 50 to 1. In certain embodiments, the disease or condition may be, but is not limited to: seizure disorders such as epilepsy, treatment-resistant epilepsy, Dravet Syndrom, Lennox-Gestaut Syndrome; spasticity disorders such as multiple sclerosis; neurodegenerative disorders such as Parkinson's Disease, Alzheimer's Disease, Huntington's Disease or amyloid lateral sclerosis (ALS); proliferative diseases such as cancer; dermatological conditions such as psoriasis; mental health conditions such as post-traumatic stress disorder (PTSD), insomnia, anxiety, depression, or schizophrenia; respiratory diseases such as chronic obstructive pulmonary disorder (COPD). Methods of the disclosure include providing compositions of the disclosure to a patient or subject in need thereof

The following examples are provided for illustrative purposes, and are not intended to limit the scope of the disclosure.

### ILLUSTRATIVE WORKING EXAMPLE

### Example 1—TN-501G Drug Product

### Tilray Cannabis Extract—Active Substance Standardized to 100 mg/mL CBD; 2 mg/mL THC as an Oral Solution

### Manufacturing and Regulatory Overview

The overall manufacturing process involves the cultivation of Cannabis sativa L. as the starting material for the production of the Drug Product. The cannabis is then subjected to a crude extraction process to produce the Crude Extract. The final step in the process is the formulation of the Crude Extract into Drug Product by dissolving in a suitable excipient.

### Raw Material

### Manufacturing

Cannabis sativa L. strains are grown indoors in a facility established with optimized environmental conditions that are controlled and monitored to assure the production of cannabis to the highest standards of chemical content and microbial purity. The material is milled before extraction to maximize extraction efficiency and is then subjected to a fluid extraction process.

### Storage

Milled cannabis is stored in approximately 1 kg quantities in heat sealed polyethylene bags. Labelled storage is room temperature. Material older than six months is subjected to reanalysis prior to being released for extraction.

### Crude Extract

### Manufacturing

Crude extraction involves a proprietary fluid based extraction. Extraction materials comply with USP/EP requirements.

The extraction continues with a decarboxylation heating cycle and subsequent fluid and moisture removal by use of rotary evaporation. The final crude extract is assayed for potency.

### Storage

Crude extract is stored in 1 L Type III amber glass bottles with a polypropylene cap. Labelled storage is refrigeration. Material older than three months is subjected to reanalysis prior to being released for purification.

### Cannabinoid Purification

### Manufacturing

The crude extract is further processed to isolate purified cannabidiol (CBD) and delta-9-tetrahydrocannabinol (THC). The crude extract is subjected to a combination of preparatory liquid chromatography and selective crystallization. Subsequent fluid and moisture removal is executed by the use of rotary evaporation. Purified CBD or THC is assayed for purity and impurities prior to formulation into Drug Product.

### Drug Product

### Manufacturing

The purified cannabinoid substances are dissolved in excipient(s) which are pharmaceutical grade or GRAS approved under conditions that ensure full dissolution of the extract. TN-501G Drug Product is formulated in grapeseed oil. In-process confirmation of potency is followed by final filling into 25 ml HDPE bottles.

### Specifications

Drug Product meets the following specifications:

| Test | Acceptance Criteria | |
|---|---|---|
| Chemistry: | | |
| Appearance | Clear yellow solution | |
| Identification - CBD or THC | Retention times correspond to | |
| | that in a standard chromatogram | |
| Assay - CBD or THC | 90.0%-100% label claim | |
| Assay - CBD | 90.0-110.0 | mg/mL |
| Assay - THC | 1.9-2.1 | mg/mL |

| | |
|---|---|
| Fill weight | 23.0 g/bottle ± 5% (21.85 g-24.15 g) |
| Residual Solvent | NMT 0.5% |
| Microbiology: | |
| Total Count | NMT 1,000 CFU/g |
| Yeast & Molds | NMT 100 CFU/g |
| Salmonella | Negative 10/g |
| Staphylococcus aureus | Negative/g |
| E. coli | Negative/g |
| Pseudomonas aeruginosa | Negative/g |

Testing conducted in accordance with compendial methods or internally validated methods.

### Example 2

Background. A study was conducted to investigate dosing and tolerability of add-on (i.e. in addition to their standard anti-epileptic therapy) Cannabidiol in children between 12 months and 18 years with treatment-resistant epilepsy due to Dravet syndrome. Dravet syndrome is a devastating syndrome which causes medication resistant epilepsy associated with significant cognitive morbidity and frequent seizures. In addition to the significant morbidity, children with refractory epilepsy are at risk of seizure-related mortality and Sudden Unexpected Death in an Epilepsy Patient (SUDEP). The risk of SUDEP in treatment resistant epilepsy is 1 in 150. The current armory of anti-epileptic drugs, dietary therapy and the Vagal Nerve Stimulator (VNS) device are not always successful in achieving seizure control. The healthcare costs of childhood-onset treatment resistant epilepsy are well established. In Dravet syndrome the severity of the epilepsy often requires frequent emergency department attendances, and hospital admissions.

The availability of a treatment to reduce the seizure frequency in children with Dravet syndrome (thus reducing hospital admissions) and to provide the chance of improved long term cognitive outcome, hence reducing the need for lifelong neurodisability service availability, would lead to significant savings in healthcare expenditure.

In addition, it is scientifically plausible that this study product would have efficacy in treating seizures in Dravet syndrome. In 80% of cases of Dravet syndrome a causative genetic mutation is detected in a Sodium channel coding gene (SCN1a mutation). Cannabinoids CBD and CBG have been shown to be potent sodium channel blockers in both human and animal models.

Formulation. Tilray TIL-TC150 is formulated with THC and CBD in grape seed oil at strengths of 2 mg/ml and 100 mg/ml, respectively. The product is formulated with standard pharmaceutical excipients.The active ingredients in TIL-TC150 Oil are THC and CBD, present in a 1:50 ratio. These active ingredients are derived from Cannabis sativa L. strains produced by Tilray, a federally-licensed producer and distributor of medical cannabis under Health Canada's Marijuana for Medical Purposes Regulations.

CBD is highly lipid soluble and is a potent inhibitor of CYP2B6, CYP2C8, CYP2C9, CYP2C19, and CYP3A4. The effects of other concomitant drug levels metabolized by these enzyme systems are not known. CBD is excreted in the urine and feces. The plasma peaks vary significantly between individuals but are typically between 1 to 2 hours. The CBD levels are detected up to 8 hours post administration.

Cannabinoids are thought to lead to increases in systolic BP over time and decreases in diastolic BP with noted heart rate increase. However, these effects are likely due to elevated THC content and many of the adverse effects are considered in keeping with the psychoactive THC effects. Mixtures of cannabinoids are less psychoactive than pure THC. Therefore, a mixture of other cannabinoids (terpenoids, etc.) may also reduce any potential psychoactive issues.

The following are the most common side effects of AEDs and/or high-THC cannabis. However, since the compositions described herein are very low in THC it is contemplated that these will be minor or rare:
a. Dizziness, irritability, ataxia, blurred vision, diplopia vision, rashes, and motor incoordination/falls.
b. Gastrointestinal (anorexia, loss of appetite, nausea, vomiting, and weight loss). It is contemplated herein that the low level of these gastrointestinal effects is due to the carrier oil, regardless such side effects are rare with the compositions described herein.
c. Psychiatric and behavioural adverse reactions such as aggression, hostility, irritability, anger, and homicidal ideation/threats.

The compositions described herein provide an optimal CBD dose while lowering the THC dose, resulting in superior performance as compared to prior art formulations.

The dosing titration protocol of up to 16 mg/kg/day of CBD is to reduce the frequency of side effects reported in a patient population. Safety and tolerability reviews were conducted by regular clinical evaluations at baseline, every 2 weeks for the first month, monthly for 4 months (to interim outcome stage) then once every 3 months thereafter (for those choosing to continue therapy). CBD were started at 2 mg/kg/day CBD and titrated slowly by 2 mg/kg/day CBD every 7 days until 16 mg/kg/day CBD is reached (or maximal tolerated dose clinically). Patients were also assessed for concomitant AED levels at baseline and maximal tolerated CBD dose. These concomitant AEDs can be adjusted as necessary based on the level and signs/symptoms of toxicity or decline in seizure control.

Dosing safety was measured by blood work evaluation of renal, hematologic and hepatic function and of AED levels, by parent/caregiver report, by physician assessment, and by assessing caregiver reported tolerability and the Pediatric Epilepsy Side Effects Questionnaire (PESQ).

Efficacy can be assessed by assessing changes from baseline in: seizure frequency using a parent-reported diary; the frequency of use of rescue medications, the frequency of status epilepticus resulting in hospital admission. Seizure frequency measurements can also include a 24-hour ambulatory EEG study which measures the percentage change in electrographic seizure frequency and the percentage change in interictal activity (e.g., using spike detection software).

Patients were monitored for a 4 week period prior to being administered a composition described herein. Following the pre-intervention assessment of baseline seizure frequency, the participant commenced the TIL-TC150 product described herein. The initial dose is 2 mg/kg/day CBD divided twice daily with weekly titration. Study treatment was increased each week as tolerated by 2 mg/kg/day CBD. Week 8 can be the last increase if the participant increases by this schedule. The maximal dose is 16/mg/kg/day CBD.

Once 16 weeks of therapy have been completed, participants entered a 4 week interim analysis period, during which time they continue the TIL-TC150 at an unchanged dose and a 4 week seizure diary and a 24 hour ambulatory EEG recording were performed and compared with the pre-intervention seizure frequency. For those participants who chose to continue TIL-TC150 therapy after the initial intervention period, they were followed by the investigator team in clinic at 3 monthly intervals to assess for any tolerability and safety issues that arise with more prolonged therapy. They can be followed to 64 weeks.

Participants can be taking concomitant AED, perhaps 1-4 AEDs. In addition, they may have a VNS device inserted or be on the ketogenic diet.

It is contemplated that treatment according to the methods described herein reduce seizure frequency due to the addition of a high CBD low THC plant extract option to the patient's current AED regimen.

The compositions described herein can be administered orally.

### Results

The study followed 19 patients, who achieved TIL-TC150 doses of 7-16 mg/kg/day of CBD by the completion of the study. The mean dose was 13.3 mg/kg/day and the median dose was 14 mg/kg/day. Forty-five percent of the patients (i.e. nine of the 19) reached the target dose of 16 mg/kg/day of CBD. Under the treatment regimen described above, 3 of 19 patients saw 90% reduction in seizures. 9 of 19 patients saw 50-90% reduction and 7 of 19 saw less than 50% reduction of seizures. Two of 19 were seizure free at week 20 while 7 of 19 were seizure free for at least 4 consecutive weeks during the study period. The number of clinically apparent seizures in a 4 week period (excluding eyelid myoclonus and myoclonic jerks) were reduced as shown in Tables 1 and 2. The therapy also demonstrated a significant impact on the quality of life. Using the QOLCE survey, the group showed a change in QOLCE score over the study period from a mean of 39.60 to 46.02, an improvement of 16.21%.

The side effects of the treatment were minimal. Twelve patients showed no significant change in bloodwork, with the remaining patients showing only transient or isolated liver abnormalities. In 8 of 19 patients, side effects were transient or resolved. The excellent side effect profile of the therapy was also reflected in the PESQ (Pediatric Side Effects Questionnaire) scores, which dropped 51.52% from a mean of 9.69 at baseline to 14.69 at week 16 of treatment. Fourteen of the patients elected to continue the treatment beyond 20 weeks.

All 19 of the patients were taking additional AEDs. The number of AEDs per patient ranged from 1 to 4 with a mean of 2.9. The additional AEDs included clobazam (in 14 patients) and valproic acid (in 12 patients).

TABLE 1
Seizure number at Baseline and Primary Endpoint

| Calculation | Baseline (week −4 to 0) | Primary Endpoint (week 16-20) |
|---|---|---|
| Mean | 31.32 | 16.47 |
| SD | 34.98 | 24.09 |
| Range | 1-137 | 0-100 |

TABLE 2
Percentage change from baseline

| Seizure reduction | Number of participants (%) |
|---|---|
| >90% | 3 (16%) |
| 50-90% | 9 (47%) |
| Less than 50% | 7 (37%) |

### Conclusion

While various embodiments have been described and illustrated herein, those of skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the disclosed teachings is/are used. Those skilled in the art will recognize, or be able to ascertain using no more than routine experimentation, equivalents to the specific embodiments described herein. It is, therefore, to be understood that the foregoing embodiments are presented by way of example only and that, within the scope of the disclosure and equivalents thereto; embodiments may be practiced otherwise than as specifically described. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments can be implemented in any of numerous ways. Also, various inventive concepts may be embodied as one or more methods, of which examples have been provided. The acts performed as part of the method may be ordered in any suitable way. Accordingly, embodiments may be constructed in which acts are performed in an order different than discussed, which may include performing some acts simultaneously, even though discussed as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein, unless clearly indicated to the contrary, should be understood to mean “at least one.”

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” shall have its ordinary meaning as used in the field of patent law.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

All transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A pharmaceutical composition comprising cannabidiol (CBD) and delta-9-tetrahydrocannabinol (THC) at a ratio of from about 40:1 to about 60:1.

2. The composition of claim 1, wherein the CBD:THC ratio is from about 45:1 to about 55:1.

3. The composition of claim 1, wherein the CBD:THC ratio is about 50:1.

4. The composition of claim 1, wherein the CBD:THC ratio is 50:1.

5. The composition of claim 1, wherein the CBD and THC are formulated in grape seed oil.

6. The composition of claim 1, wherein the CBD and THC are obtained from Cannabis sativa L.

7. The composition of claim 1, wherein the CBD and THC are provided at concentrations of about 100 mg/mL and 2 mg/mL respectively.

8. The composition of claim 1, further comprising one or more pharmaceutically acceptable carriers or excipients.

9. A method of treating pediatric epilepsy in a subject in need thereof, the method comprising administering the composition of claim 1 to the subject.

10. The method of claim 9, wherein the composition is administered twice daily.

11. The method of claim 9, wherein the composition is administered orally or by inhalation.

12. (canceled)

13. The method of claim 9, wherein the subject is administered the composition at a dosage of from about 1 mg/kg/day of CBD to about 18 mg/kg/day of CBD.

14. The method of claim 9, wherein the subject is administered the composition at a dosage of from about 5 mg/kg/day of CBD to about 18 mg/kg/day of CBD.

15. The method of claim 9, wherein the subject is administered the composition at a dosage of from about 7 mg/kg/day of CBD to about 16 mg/kg/day of CBD.

16. The method of claim 9, wherein the subject is administered the composition at a dosage of from 2 mg/kg/day of CBD to 16 mg/kg/day of CBD.

17. The method of claim 9, wherein the subject is administered the composition at an increasing dosage of the composition, wherein the increase is about 2 mg/kg/day of CBD every 7 days.

18. The method of claim 9, wherein a maximal dosage of the CBD administered to the subject is from about 13 mg/kg/day of CBD to about 14.5 mg/kg/day of CBD.

19. The method of claim 9, wherein a maximal dosage of the CBD administered to the subject is about 16 mg/kg/day of CBD.

20. The method of claim 9, wherein the subject is administered at least one further concomitant antiepileptic drug (AED).

21. The method of claim 9, whereby the treatment reduces the frequency or severity of seizures.

22.-41. (canceled)
